(19) United States
(12) Patent Application Publication
(10) Pub. No.: US 2026/0309098 A1
(43) Pub. Date: Oct. 8, 2026

(54) GAS TURBINE SYSTEM AND METHOD FOR MODIFYING GAS TURBINE SYSTEM

(71) Applicant: IHI Corporation, Tokyo (JP)

(72) Inventors: Koukichi ABE, Tokyo (JP); Kodai IWANAGA, Tokyo (JP); Masahiro UCHIDA, Tokyo (JP); Shintaro ITO, Tokyo (JP)

(73) Assignee: IHI Corporation, Tokyo (JP)

(21) Appl. No.: 19/682,754

(22) Filed: May 20, 2026

Related U.S. Application Data
(63) Continuation of application No. PCT/JP2024/038640, filed on Oct. 30, 2024.

(30) Foreign Application Priority Data
Nov. 22, 2023 (JP) 2023-198470

(51) Int. Cl. F02C 9/28

(52) U.S. Cl. CPC F02C 9/28

(57) ABSTRACT

A gas turbine system includes a gas turbine that includes a combustor, a first line that supplies a first fuel to the combustor, a second line that supplies a second fuel of which flame temperature is lower than that of the first fuel to the combustor, a first adjuster that adjusts a first flow rate of the first fuel flowing into the combustor, a second adjuster that adjusts a second flow rate of the second fuel flowing into the combustor, and a controller that is communicatively connected to the first adjuster and the second adjuster. The controller is configured to execute calculating a flow rate ratio indicating a ratio between the first flow rate and the second flow rate, and controlling the first adjuster and the second adjuster based on the flow rate ratio.

### CROSS REFERENCE TO RELATED APPLICATIONS

This application is a continuation application of International Application No. PCT/JP2024/038640, filed on Oct. 30, 2024, which claims priority to Japanese Patent Application No. 2023-198470 filed on Nov. 22, 2023, the entire contents of which are incorporated herein by reference.

### BACKGROUND ART

### Technical Field

The present disclosure relates to a gas turbine system and a method for modifying a gas turbine system.

Maximum output of a gas turbine may be set such that gas temperature at an inlet of a turbine does not exceed heat-resistant temperature of the inlet of the turbine. However, the gas temperature at the inlet of the turbine can be extremely high and difficult to detect. Accordingly, for example, Non-Patent Literature 1 discloses a method for indirectly determining gas temperature at an inlet of a turbine based on the gas temperature at an outlet of the turbine.

### CITATION LIST

### Non-Patent Literature

Non-Patent Literature 1: Masatoshi Kato et al., “Gas Turbine Controls and Operation,” Hitachi Review, March 1969 issue, p. 62-69

### SUMMARY

### Technical Problem

To promote the use of carbon-neutral fuels, substitution with carbon-free fuels such as ammonia is being considered. Flame temperature of a carbon-free fuel may be lower than that of a hydrocarbon fuel. Accordingly, for example, when such a carbon-free fuel is combusted in co-firing with a hydrocarbon fuel in a gas turbine, temperature of combustion gas decreases compared to when only the hydrocarbon fuel is combusted to achieve the same output. Consequently, gas temperature at an inlet of a turbine also decreases. In this case, even if the output of the gas turbine is increased, the gas temperature at the inlet of the turbine does not exceed a heat-resistant temperature of the inlet of the turbine. Accordingly, in this case, the maximum output of the gas turbine can be increased compared to when only hydrocarbon fuel is combusted. However, in many cases, the same maximum output as when combusting only hydrocarbon fuel is used.

The present disclosure aims to provide a gas turbine system and a method for modifying a gas turbine system that can increase maximum output.

### Solution to Problem

A gas turbine system according to one aspect of the present disclosure includes a gas turbine that includes a combustor, a first line that is fluidly connected to the combustor and that supplies a first fuel to the combustor, a second line that is fluidly connected to the combustor and that supplies a second fuel of which flame temperature is lower than that of the first fuel to the combustor, a first adjuster that is provided in the first line and that adjusts a first flow rate of the first fuel flowing into the combustor, a second adjuster that is provided in the second line and that adjusts a second flow rate of the second fuel flowing into the combustor, and a controller that is communicatively connected to the first adjuster and the second adjuster, the controller being configured to execute calculating a flow rate ratio indicating a ratio between the first flow rate and the second flow rate, and controlling the first adjuster and the second adjuster based on the flow rate ratio.

Controlling the first adjuster and the second adjuster may include determining maximum output of the gas turbine based on the flow rate ratio, obtaining output of the gas turbine, and controlling the first adjuster and the second adjuster to adjust the first flow rate and the second flow rate such that the obtained output of the gas turbine does not exceed the maximum output.

The gas turbine system may include a temperature sensor that measures temperature of intake air to a compressor of the gas turbine, the controller may be communicatively connected to the temperature sensor, and determining the maximum output of the gas turbine may include adjusting the maximum output of the gas turbine based on measurement data from the temperature sensor.

The gas turbine system may include a first analyzer that measures composition of the first fuel, the controller may be communicatively connected to the first analyzer, and determining the maximum output of the gas turbine may include adjusting the maximum output of the gas turbine based on measurement data from the first analyzer.

The gas turbine system may include a second analyzer that measures composition of the second fuel, the controller may be communicatively connected to the second analyzer, and determining the maximum output of the gas turbine may include adjusting the maximum output of the gas turbine based on measurement data from the second analyzer.

A method for modifying a gas turbine system according to another aspect of the present disclosure includes preparing a program for controlling the gas turbine system, the gas turbine system including a gas turbine that includes a combustor, a first line that is fluidly connected to the combustor and that supplies a first fuel to the combustor, a second line that is fluidly connected to the combustor and that supplies a second fuel of which flame temperature is lower than that of the first fuel to the combustor, a first adjuster that is provided in the first line and that adjusts a first flow rate of the first fuel flowing into the combustor, a second adjuster that is provided in the second line and that adjusts a second flow rate of the second fuel flowing into the combustor, and a controller communicatively connected to the first adjuster and the second adjuster, the program causing a computer to execute calculating a flow rate ratio indicating a ratio between the first flow rate and the second flow rate, and controlling the first adjuster and the second adjuster based on the flow rate ratio, and installing the program on the controller.

### Effects

According to the present disclosure, maximum output of a gas turbine can be increased.

### BRIEF DESCRIPTION OF DRAWINGS

FIG. 1 is a schematic diagram showing a gas turbine system according to a first embodiment.

FIG. 2 is a flowchart showing an example of processing by a controller.

FIG. 3 is a graph showing a relationship between output of a gas turbine and gas temperature at an inlet of a turbine.

FIG. 4 is a graph showing an example of processing by the controller.

FIG. 5 is a graph showing another example of processing by the controller.

FIG. 6 is a graph showing yet another example of processing by the controller.

FIG. 7 is a schematic diagram showing a gas turbine system according to a second embodiment.

### DESCRIPTION OF EMBODIMENTS

Embodiments of the present disclosure will be described in detail below with reference to the accompanying drawings. Specific dimensions, materials, and numerical values described in the embodiments are merely examples for better understanding, and do not limit the present disclosure unless otherwise specified. In this specification and the drawings, duplicate explanations are omitted for elements having substantially the same functions and configurations by assigning the same sign. Furthermore, elements not directly related to the present disclosure are omitted from the figures.

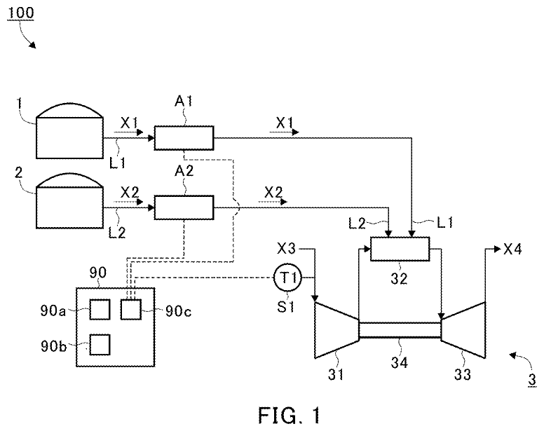

FIG. 1 is a schematic diagram showing a gas turbine system 100 according to a first embodiment. In the present disclosure, the gas turbine system may simply be referred to as the “system.” For example, the system 100 includes a first tank (first fuel supply source) 1, a second tank (second fuel supply source) 2, a first adjuster A1, a second adjuster A2, a gas turbine 3, a temperature sensor S1, and a controller 90. The system 100 may further include other components. Furthermore, the gas turbine 3 includes a compressor 31, a combustor 32, and a turbine 33. The gas turbine 3 may further include other components.

The first tank 1 stores first fuel X1. For example, the first fuel X1 may be a hydrocarbon fuel such as natural gas. The first tank 1 is connected to a first line L1. For example, the first line L1 includes one or more pipes. The first line L1 is connected to the combustor 32. The first tank 1 supplies the first fuel X1 to the combustor 32 via the first line L1. For example, the first line L1 may include a compressor or a pump (not shown) for supplying the first fuel X1. For example, in other embodiments, an apparatus for producing the first fuel may be used as the first fuel supply source instead of the first tank 1.

The second tank 2 stores a second fuel X2. Flame temperature of the second fuel X2 is lower than that of the first fuel. In the present disclosure, the “flame temperature” may be defined as “adiabatic flame temperature.” For example, the second fuel X2 may be ammonia. For example, the second tank 2 may store liquid ammonia or gaseous ammonia. The second tank 2 is connected to a second line L2. For example, the second line L2 includes one or more pipes. The second line L2 is connected to the combustor 32. The second tank 2 supplies the second fuel X2 to the combustor 32 via the second line L2. For example, the second line L2 may include a compressor or a pump (not shown) for supplying the second fuel X2. Furthermore, for example, when the second tank 2 stores liquid ammonia, the second line L2 may include a vaporizer (not shown). For example, in other embodiments, an apparatus for producing the second fuel may be used as the second fuel supply source instead of the second tank 2.

The first adjuster A1 is provided in the first line L1 between the first tank 1 and the combustor 32. The first adjuster A1 adjusts a flow rate of the first fuel X1 flowing through the first line L1, i.e., a first flow rate F1 of the first fuel X1 flowing into the combustor 32. For example, the first adjuster A1 may include a flow controller capable of measuring the first flow rate F1 and adjusting the first flow rate F1. Alternatively, or additionally, for example, the first adjuster A1 may include a flow meter and at least one valve. The first adjuster A1 is communicatively connected to the controller 90 and transmits measurement data to the controller 90. The controller 90 is configured to adjust the first flow rate F1 by controlling the first adjuster A1.

The second adjuster A2 is provided in the second line L2 between the second tank 2 and the combustor 32. The second adjuster A2 adjusts a flow rate of the second fuel X2 flowing through the second line L2, i.e., a second flow rate F2 of the second fuel X2 flowing into the combustor 32. For example, the second adjuster A2 may include a flow controller capable of measuring the second flow rate F2 and adjusting the second flow rate F2. Alternatively, or additionally, for example, the second adjuster A2 may include a flow meter and at least one valve. The second adjuster A2 is communicatively connected to the controller 90 and transmits measurement data to the controller 90. The controller 90 is configured to adjust the second flow rate F2 by controlling the second adjuster A2.

The compressor 31 pressurizes sucked-in air (intake air) X3. For example, the air X3 may be ambient air around the compressor 31. The pressurized air X3 is supplied to the combustor 32. The combustor 32 combusts mixed gas containing the first fuel X1 from the first tank 1, the second fuel X2 from the second tank 2, and the air X3 from the compressor 31. Combustion gas X4 from the combustor 32 is supplied to the turbine 33. The compressor 31 and the turbine 33 are connected to each other by a shaft 34.

The combustion gas X4 passes through an impeller in the turbine 33 and rotates the impeller along with the shaft 34. For example, the shaft 34 may be connected to a generator (not shown), and rotational force of the shaft 34 may be used for power generation. In other embodiments, the rotational force of the shaft 34 may be used by other equipment. Furthermore, the rotational force of shaft 34 is used to pressurize the air X3 in the compressor 31. The combustion gas X4 passing through the turbine 33 may be used in various equipment, such as a boiler.

The temperature sensor S1 measures temperature T1 of the air X3 sucked into the compressor 31. For example, the temperature sensor S1 may be provided in piping connected to the compressor 31. The temperature sensor S1 is communicatively connected to the controller 90 and transmits the temperature T1 to the controller 90.

The controller 90 controls the entire system 100 or a portion thereof. For example, the controller 90 may be configured by one or more PCs. The controller 90 includes components such as a processor 90a, a memory 90b, and a connector 90c, and these components are connected to each other via buses. For example, the processor 90a includes a CPU (Central Processing Unit), etc. For example, the memory device 90b includes a hard disk, ROM where programs are stored, and RAM serving as a work area, etc. The controller 90 is communicatively connected via the connector 90c to each component of the system 100 by wire or wirelessly. For example, the controller 90 may further include other components such as a display like an LCD or a touch panel, and an input device like a keyboard, buttons, or a touch panel. For example, the following operations of the controller 90 may be realized by executing programs stored in the memory 90b by the processor 90a.

Next, operation of the system 100 will be described.

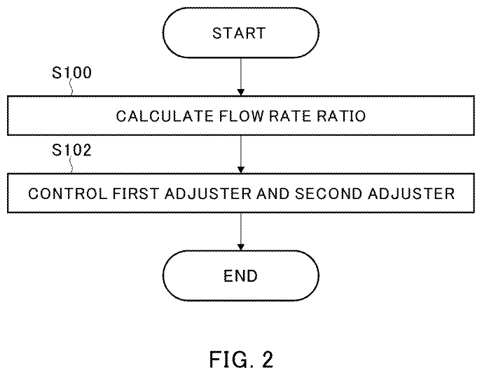

FIG. 2 is a flowchart showing an example of processing by the controller 90. For example, the processing shown in FIG. 2 may be periodically repeated during operation of the gas turbine system 100.

The processor 90a calculates a flow rate ratio R (step S100). In the present disclosure, the “flow rate ratio” indicates a ratio between the first flow rate F1 of the first fuel X1 and the second flow rate F2 of the second fuel X2. For example, in the present embodiment, the flow rate ratio R means a proportion of the second flow rate F2 relative to the total flow rate of the first flow rate F1 and the second flow rate F2 (R=F2/(F1+F2)×100 (%)). In other embodiments, the flow rate ratio R may mean a proportion of the first flow rate F1 relative to the total flow rate of the first flow rate F1 and the second flow rate F2 (R=F1/(F1+F2)×100 (%)). The processor 90a can receive the first flow rate F1 and the second flow rate F2 from the first adjuster A1 and the second adjuster A2, respectively.

The processor 90a controls the first adjuster A1 and the second adjuster A2 based on the flow rate ratio R (step S102), and terminates the operation shown in FIG. 2. Specifically, the processor 90a controls the first adjuster A1 and the second adjuster A2 as described below.

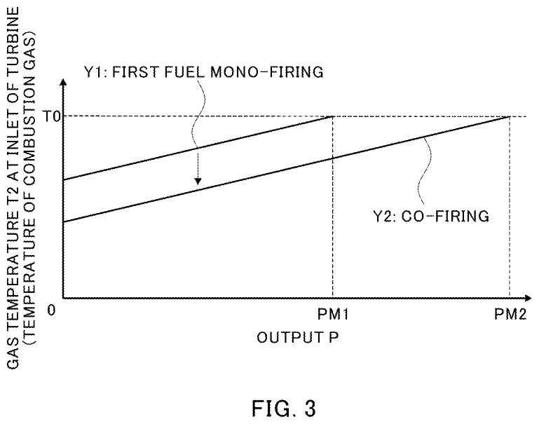

FIG. 3 is a graph showing a relationship between output P of the gas turbine 3 and gas temperature T2 at an inlet of the turbine 33. In FIG. 3, a horizontal axis indicates the output P of the gas turbine 3, and a vertical axis indicates the gas temperature T2 at the inlet of the turbine 33, i.e., temperature of the combustion gas X4 from the combustor 32.

T0 indicates a heat-resistant temperature at the inlet of the turbine 33. For example, the heat-resistant temperature T0 may be determined by factors such as materials used at the inlet of the turbine 33. In the turbine 33, the temperature T2 of the combustion gas X4 is the highest at before passing through the impeller, i.e., the highest at the inlet. Accordingly, the heat-resistant temperature T0 at the inlet of the turbine 33 is considered.

In FIG. 3, a line Y1 indicates the temperature T2 of when only the first fuel X1 is combusted, i.e., when the flow rate ratio R=0%. A line Y 2 indicates the temperature T2 of when the first fuel X1 and the second fuel X2 are co-fired. As described above, the adiabatic flame temperature of the second fuel X2 is lower than that of the first fuel X1. Accordingly, when comparing under the same output conditions, the temperature T2 of the combustion gas X4 of when the first fuel X1 and the second fuel X2 are co-fired (line Y2) is lower than that of when only the first fuel X1 is combusted (line Y1). In other words, the line Y2 is lower than the line Y1. In the present embodiment, the line Y2 decreases (slides downward) as the flow rate ratio R increases.

As indicated by the lines Y1 and Y2, the temperature T2 increases as the output P increases. The gas turbine 3 has to be operated such that the temperature T2 of the combustion gas X4 does not exceed the heat-resistant temperature T0. Accordingly, a maximum output PM1 of when only the first fuel X1 is combusted can be determined as a point where the line Y1 intersects the heat-resistant temperature T0. Similarly, a maximum output PM2 of when the first fuel X1 and the second fuel X2 are co-fired can be determined as a point where the line Y2 intersects the heat-Resistant temperature T0.

As shown in FIG. 3, the maximum output PM2 of when the first fuel X1 and the second fuel X2 are co-fired can be set higher than the maximum output PM1 of when only the first fuel X1 is combusted. Accordingly, when using the second fuel X2 such as ammonia, the maximum output PM2 can be increased compared to when only the first fuel X1 such as natural gas is combusted. The maximum output PM2 varies depending on the flow rate ratio. Specifically, in the present embodiment, the maximum output PM2 increases as the flow rate ratio R increases.

For example, the controller 90 may store a map that shows a relationship between the flow rate ratio R and the maximum output PM of the gas turbine 3 in the memory 90b. For example, the processor 90a may read the maximum output PM corresponding to the flow rate ratio R calculated in step S100 from the map. Alternatively, or additionally, the controller 90 may store a formula that shows a relationship between the flow rate ratio R and the maximum output PM of the gas turbine 3 in the memory 90b. For example, the processor 90a may calculate the maximum output PM by substituting the flow rate ratio R calculated in step S100 into the formula.

Additionally, the controller 90 may adjust the maximum output PM based on the temperature T1 from the temperature sensor S1. Generally, as the temperature T1 of the air X3 sucked into the compressor 31 increases, the output of the gas turbine 3 decreases, and as the temperature T1 decreases, the output increases. Accordingly, for example, the processor 90a may adjust the maximum output PM such that the maximum output PM decreases as the temperature T1 increases and the maximum output PM increases as the temperature T1 decreases.

For example, the controller 90 may store a map that shows a relationship between the flow rate ratio R, the temperature T1, and the maximum output PM of the gas turbine 3 in the memory 90b. For example, the processor 90a may read the maximum output PM corresponding to the flow rate ratio R calculated in step S100 and the temperature T1 received from the temperature sensor S1 from the map. Alternatively, or additionally, the controller 90 may store a formula that shows a relationship between the flow rate ratio R, the temperature T1, and the maximum output PM of the gas turbine 3 in the memory 90b. For example, the processor 90a may calculate the maximum output PM by substituting the flow rate ratio R calculated in step S100 and the temperature T1 received from the temperature sensor S1 into the formula.

The processor 90a obtains the current output P of the gas turbine 3. For example, the controller 90 may be communicatively connected to the gas turbine 3 and may calculate the output P of the gas turbine 3 based on various measurement data transmitted from the gas turbine 3. The method for obtaining the output P of the gas turbine 3 is not limited thereto.

The processor 90a controls the first adjuster A1 and the second adjuster A2 to adjust the first flow rate F1 and the second flow rate F2 such that the obtained output P of the gas turbine 3 does not exceed the determined maximum output PM.

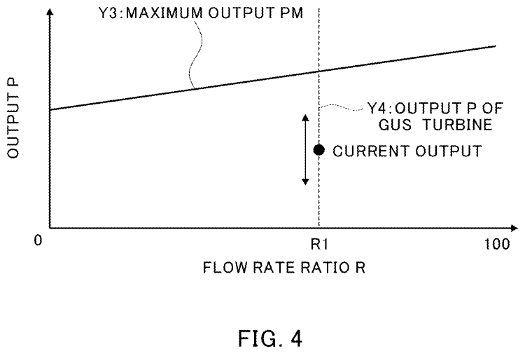

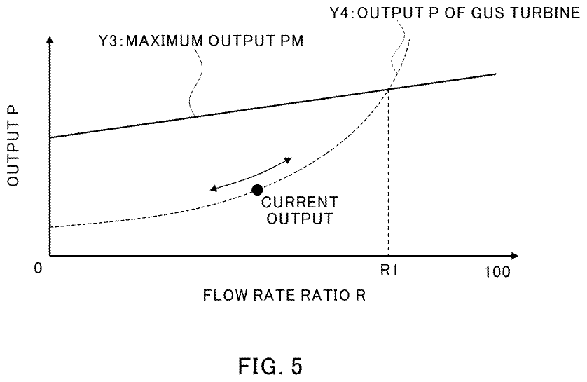

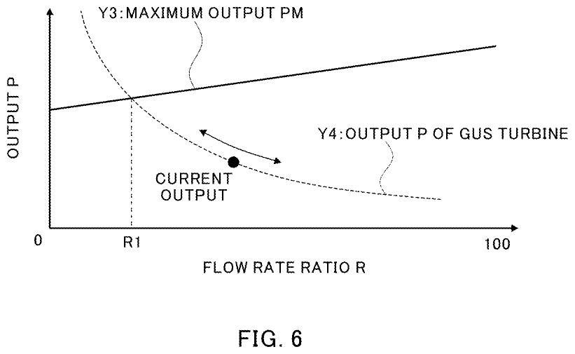

FIG. 4 is a graph showing an example of processing by the controller 90. In FIG. 4 and the following FIGS. 5 and 6, a horizontal axis indicates the flow rate ratio R, a vertical axis indicates the output P of the gas turbine 3, a solid line Y3 indicates the maximum output PM of the gas turbine 3 determined as described above, and a dashed line Y4 indicates the output P of the gas turbine 3.

For example, the gas turbine system 100 may be required to operate with the flow rate ratio R1 fixed for various reasons. In this state, the maximum output PM is also fixed at a constant value. In this case, the processor 90a controls the first adjuster A1 and the second adjuster A2 to adjust the first flow rate F1 and the second flow rate F2 so that the output P of the gas turbine 3 does not exceed the fixed maximum output PM and the flow rate ratio R is maintained at a constant value R1.

FIG. 5 is a graph showing another example of processing by the controller 90.

For example, the gas turbine system 100 may be required to operate with the first flow rate F1 of the first fuel X1 fixed for various reasons. In this case, only the second flow rate F2 of the second fuel X2 is adjusted. In this state, as shown by the dashed line Y4, the output P of the gas turbine increases as the flow rate ratio R increases, in the present embodiment. In this case, the processor 90a only controls the second adjuster A2 to only adjust the second flow rate F2 so that the flow rate ratio R does not exceed the value R1 corresponding to the intersection point between the line Y3 and the dashed line Y4.

FIG. 6 is a graph showing yet another example of processing by the controller 90.

For example, the gas turbine system 100 may be required to operate with the second flow rate F2 of the second fuel X2 fixed for various reasons. In this case, only the first flow rate F1 of the first fuel X1 is adjusted. In this state, as shown by the dashed line Y4, the output P of the gas turbine increases as the flow rate ratio R decreases, in the present embodiment. In this case, the processor 90a only controls the first adjuster A1 to only adjust the first flow rate F1 so that the flow rate ratio R does not fall below the value R1 corresponding to the intersection point between the line Y3 and the dashed line Y4.

The controller 90 may also store a compressor map showing a relationship between the flow rate of the air X3 and pressure within the compressor 31, showing a boundary line for causing surging and a boundary line for causing choking. Furthermore, the controller 90 may store a turbine map showing a relationship between the flow rate of the combustion gas X4 and pressure within the turbine 33, showing a boundary line for causing choking. The controller 90 may control the system 100 based on these maps so as not to cause surging and choking.

The system 100 as described above includes the gas turbine 3 that includes the combustor 32, the first line L1 that is fluidly connected to the combustor 32 and that supplies the first fuel X1 to the combustor 32, the second line L2 that is fluidly connected to the combustor 32 and that supplies the second fuel X2 of which flame temperature is lower than that of the first fuel X1 to the combustor 32, the first adjuster A1 that is provided in the first line L1 and that adjusts the first flow rate F1 of the first fuel X1 flowing into the combustor 32, the second adjuster A2 that is provided in the second line L2 and that adjusts the second flow rate F2 of the second fuel X2 flowing into the combustor 32, and the controller 90 that is communicatively connected to the first adjuster A1 and the second adjuster A2. The controller 90 is configured to execute calculating the flow rate ratio R that indicates the ratio between the first flow rate F1 and the second flow rate F2, and controlling the first adjuster A1 and the second adjuster A2 based on the flow rate ratio R. As described above, the temperature T2 of the combustion gas X4 of when the first fuel X1 and the second fuel X2 are co-fired is lower than that of when only the first fuel X1 is combusted. Accordingly, the maximum output PM2 of the gas turbine 3 of when the first fuel X1 and the second fuel X2 are co-fired can be set higher than the maximum output PM1 of when only the first fuel X1 is combusted. Furthermore, the maximum output PM2 varies depending on the flow rate ratio R. Accordingly, by controlling the first adjuster A1 and the second adjuster A2 depending on the flow rate ratio R, the maximum output PM of the gas turbine 3 can be increased.

Furthermore, in the system 100, controlling the first adjuster A1 and the second adjuster A2 includes determining the maximum output PM of the gas turbine 3 based on the flow rate ratio R, obtaining the output P of the gas turbine 3, and controlling the first adjuster A1 and the second adjuster A2 to adjust the first flow rate F1 and the second flow rate F2 such that the obtained output P of the gas turbine 3 does not exceed the maximum output PM. According to such a configuration, it is possible to reliably prevent the current output P of the gas turbine 3 from exceeding the maximum output PM.

Furthermore, the system 100 includes the temperature sensor S1 that measures the temperature T1 of the intake air X3 to the compressor 31 of the gas turbine 3, the controller 90 is communicatively connected to the temperature sensor S1, and determining the maximum output PM of the gas turbine 3 includes adjusting the maximum output PM of the gas turbine 3 based on measurement data from the temperature sensor S1. According to such a configuration, the maximum output PM can be determined more accurately.

Furthermore, the system 100 as described above can be realized by the following method for modifying an existing gas turbine system. The method for modifying the gas turbine system includes preparing a program for controlling the gas turbine system. The gas turbine system to which this method is applied includes the gas turbine 3 that includes the combustor 32, the first line L1 that is fluidly connected to the combustor 32 and that supplies the first fuel X1 to the combustor 32, the second line L2 that is fluidly connected to the combustor 32 and that supplies the second fuel X2 of which flame temperature is lower than that of the first fuel X1 to the combustor 32, the first adjuster A1 that is provided in the first line L1 and that adjusts the first flow rate F1 of the first fuel X1 flowing into the combustor 32, the second adjuster A2 that is provided in the second line L2 and that adjusting the second flow rate F2 of the second fuel X2 flowing into the combustor 32, and the controller 90 communicatively connected to the first adjuster A1 and the second adjuster A2. Furthermore, the prepared program is configured to cause a computer to execute calculating the flow rate ratio R indicating the ratio between the first flow rate F1 and the second flow rate F2, and controlling the first adjuster A1 and the second adjuster A2 based on the flow rate ratio R. The method includes installing the prepared program on the controller 90. According to such a configuration, the maximum output PM of the gas turbine 3 can be increased in an existing gas turbine system.

Next, other embodiments will be described.

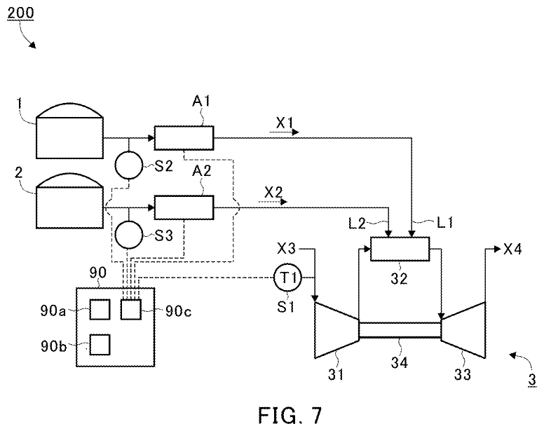

FIG. 7 is a schematic diagram showing a gas turbine system 200 according to a second embodiment. The system 200 differs from the system 100 according to the first embodiment in that the system 200 includes a first analyzer S2 and a second analyzer S3. In other configurations, the system 200 may be the same as the system 100.

The first analyzer S2 measures composition of the first fuel X1. For example, the first analyzer S2 may be provided in the first line L1. In the present embodiment, the first analyzer S2 is provided in the first line L1 between the first tank 1 and the first adjuster A1. In other embodiments, the first analyzer S2 may be provided in the first tank 1, or may be provided in the first line L1 between the first adjuster A1 and the combustor 32. For example, the first analyzer S2 may be a calorimeter. The first analyzer S2 is communicatively connected to the controller 90 and transmits measurement data to the controller 90. For example, the controller 90 may calculate a lower heating value (LHV) of the first fuel X1 based on the measurement data of the first fuel X1 from the first analyzer S2, and may calculate the flame temperature of the first fuel X1 from the lower heating value.

The second analyzer S3 measures composition of the second fuel X2. For example, the second analyzer S3 may be provided in the second line L2. In the present embodiment, the second analyzer S3 is provided in the second line L2 between the second tank 2 and the second adjuster A2. In other embodiments, the second analyzer S3 may be provided in the second tank 2, or may be provided in the second line L2 between the second adjuster A2 and the combustor 32. For example, the second analyzer S3 may be a calorimeter. The second analyzer S3 is communicatively connected to the controller 90 and transmits measurement data to the controller 90. For example, the controller 90 may calculate a lower heating value of the second fuel X2 based on the measurement data of the second fuel X2 from the second analyzer S3, and may calculate the flame temperature of the second fuel X2 from the lower heating value.

Generally, the flame temperature of a fuel may vary depending on its composition. For example, the composition of a fuel may vary depending on various factors. The flame temperatures of the first fuel X1 and the second fuel X2 affect the temperature T2 of the combustion gas X4. Accordingly, the controller 90 may adjust the maximum output PM of the gas turbine 3 in detail by considering the composition of the first fuel X1 and the composition of the second fuel X2.

For example, the controller 90 may store reference flame temperatures for the first fuel X1 and the second fuel X2 in advance in the memory 90b, and may adjust (correct) the maximum output PM based on the ratio between the calculated flame temperatures of the first fuel X1 and the second fuel X2 and the reference flame temperatures of the first fuel X1 and the second fuel X2.

Furthermore, for example, the controller 90 may store reference lower heating values for the first fuel X1 and the second fuel X2 in advance in the memory 90b, and may adjust (correct) the maximum output PM based on the ratio between the calculated lower heating values for the first fuel X1 and the second fuel X2 and the reference lower heating values for the first fuel X1 and the second fuel X2.

The method for adjusting the maximum output PM based on the measurement data from the first analyzer S2 and the second analyzer S3 is not limited thereto.

The system 200 as described above has similar effects to those of the system 100.

In particular, the system 200 includes the first analyzer S2 that measures the composition of the first fuel X1, the controller 90 is communicatively connected to the first analyzer S2, and determining the maximum output PM of the gas turbine 3 includes adjusting the maximum output PM of the gas turbine 3 based on the measurement data from the first analyzer S2. Furthermore, the system 200 includes the second analyzer S3 that measures the composition of the second fuel X2, the controller 90 is communicatively connected to the second analyzer S3, and determining the maximum output PM of the gas turbine 3 includes adjusting the maximum output PM of the gas turbine 3 based on the measurement data from the second analyzer S3. According to these configurations, the maximum output PM can be determined more accurately.

Although the embodiments of the present disclosure have been described above with reference to the accompanying drawings, the present disclosure is not limited thereto. It is obvious that a person skilled in the art can conceive of various examples of variations or modifications within the scope of the claims, which are also understood to belong to the technical scope of the present disclosure.

For example, in the above embodiment, the controller 90 determines the maximum output PM of the gas turbine 3 based on the flow rate ratio. In other embodiments, the controller 90 may not determine the maximum output PM. In this case, for example, the controller 90 may store in advance in the memory 90b a map or a formula showing a relationship between the flow rate ratio R, and the first flow rate F1 and the second flow rate F2 for obtaining the maximum output PM that corresponds to the flow rate ratio R. In this case, the controller 90 does not need to determine the maximum output PM.

Furthermore, for example, the system 200 includes both the first analyzer S2 and the second analyzer S3. In other embodiments, the system 200 may include only one of the first analyzer S2 and the second analyzer S3.

The present disclosure can promote the use of ammonia to reduce CO₂ emissions, thus contributing to Sustainable Development Goals (SDGs), Goal 7 “Ensure access to affordable, reliable, sustainable and modern energy,” for example.

What is claimed is:

1. A gas turbine system comprising:
a gas turbine that includes a combustor;
a first line that is fluidly connected to the combustor and that supplies a first fuel to the combustor;
a second line that is fluidly connected to the combustor and that supplies a second fuel of which flame temperature is lower than that of the first fuel to the combustor;
a first adjuster that is provided in the first line and that adjusts a first flow rate of the first fuel flowing into the combustor;
a second adjuster that is provided in the second line and that adjusts a second flow rate of the second fuel flowing into the combustor; and
a controller that is communicatively connected to the first adjuster and the second adjuster, the controller being configured to execute:
calculating a flow rate ratio indicating a ratio between the first flow rate and the second flow rate; and
controlling the first adjuster and the second adjuster based on the flow rate ratio.

2. The gas turbine system according to claim 1, wherein controlling the first adjuster and the second adjuster includes:
determining maximum output of the gas turbine based on the flow rate ratio;
obtaining output of the gas turbine; and
controlling the first adjuster and the second adjuster to adjust the first flow rate and the second flow rate so that the obtained output of the gas turbine does not exceed the maximum output.

3. The gas turbine system according to claim 2, comprising:
a temperature sensor that measures temperature of intake air to a compressor of the gas turbine,
wherein
the controller is communicatively connected to the temperature sensor, and
determining the maximum output of the gas turbine includes adjusting the maximum output of the gas turbine based on measurement data from the temperature sensor.

4. The gas turbine system according to claim 2, comprising:
a first analyzer that measures composition of the first fuel,
wherein
the controller is communicatively connected to the first analyzer, and
determining the maximum output of the gas turbine includes adjusting the maximum output of the gas turbine based on measurement data from the first analyzer.

5. The gas turbine system according to claim 2, comprising:
a second analyzer that measures composition of the second fuel,
wherein
the controller is communicatively connected to the second analyzer, and
determining the maximum output of the gas turbine includes adjusting the maximum output of the gas turbine based on measurement data from the second analyzer.

6. A method for modifying a gas turbine system, including:
preparing a program for controlling the gas turbine system, the gas turbine system comprising:
a gas turbine that includes a combustor;
a first line that is fluidly connected to the combustor and that supplies a first fuel to the combustor;
a second line that is fluidly connected to the combustor and that supplies a second fuel of which flame temperature is lower than that of the first fuel to the combustor;
a first adjuster that is provided in the first line and that adjusts a first flow rate of the first fuel flowing into the combustor;
a second adjuster that is provided in the second line and that adjusts a second flow rate of the second fuel flowing into the combustor; and
a controller that is communicatively connected to the first adjuster and the second adjuster,
the program causing a computer to execute:
calculating a flow rate ratio indicating a ratio between the first flow rate and the second flow rate; and
controlling the first adjuster and the second adjuster based on the flow rate ratio, and
installing the program on the controller.
